(19) United States
(12) Patent Application Publication
(10) Pub. No.: US 2003/0011743 A1
(43) Pub. Date: Jan. 16, 2003

(54) Apparatus and method for detecting eyesight direction, angle and gazing point

(76) Inventor: Boris Povlotsky, Sterling Hts, MI (US)

Correspondence Address: Boris Povlotsky, 41255 POND VIEW DR # 349, STERLING HTS, MI, 48314, US

(21) Appl. No.: 09/775,421

(22) Filed: Feb. 1, 2001

Related U.S. Application Data
(60) Provisional application No. 60/179,470, filed on Feb. 1, 2000.

(51) Int. Cl. A61B 3/14

(52) U.S. Cl. 351/209

(57) ABSTRACT

An apparatus and method for detecting eyesight direction, angle and gaze point is an optical instrument attached to supporting means (platform, helmet or headset, etc.) and positioned by hand utilizing a firearm's type sight aiming system. This procedure determines the individual's eyesight direction. The instrument's integral inclination measuring system and laser pointer determine eyesight's angle gazing point.

### CROSS-REFERENCE TO RELATED APPLICATIONS

1. Eye tracker, mounted on a helmet or headset. Foster-Miller, Inc., 350 Second Ave., Waltham, Mass. 02451

2. Eye Tracking System, Applied Science Laboratories, 175 Middlesex Turnpike, Bedford, Mass. 01730, USA

3. Ergomeasure™, a tool to adjust office furniture, including computer keyboards and monitor. Herman Miller, Inc. Zeeland, Mich., USA

4. Ergoscale™—the Office Ergonomics Solutions Kit—a tool to adjust office furniture, including computer keyboard and monitor. Ergo Design, Inc. Englewood Cliffs, N.J.

### BACKGROUND—FIELD OF THE INVENTION

This invention relates to human visual performance as well as to the engineering anthropometry.

In anthropometric theory and practice, there are many occasions where there is a need to determine eyesight and use it as a reference point. For example, a horizontal eyesight level is used as the reference point (Standard Sight Line) in anthropometric theory as eye height while sitting or standing. Designers of vehicle or truck's cabin, airliner's cockpit, etc. are using the horizontal eyesight level as the reference point to lay out the operator's workplace. According to the American National Standards Institute (ANSI), the top of the VDT-computer's monitor should be positioned at the operator's eye level.

There are many ways, methods and tools to measure human eye level. Human eyes are too sensitive to be touched by direct techniques of detecting the eye level in static or dynamic measurement. The eyes are so easily hurt by undue pressure that indirect methods, such as optical sighting, are desirable in the traditional engineering anthropometry. However, setting up a surveyor's transit is often too cumbersome and time consuming. A compromise method developed by J. A. Roebuck, Jr. is very often used. This method includes a device containing two transparent parallel planes, scribed with a grid as coordinate lines. One aligns the grid lines in the two planes and records the location of the eye on these grids, estimating the distance between the lines on the plane by balancing the offset of the more distant lines between the nearer set of lines. More modem methods are high speed anthropometric scanners for static measurements and the Eye Tracking System (U.S. Pat. No. 4,852,988) of the Applied Science Laboratories (ASL) Bedford, Mass., (www.a-s-l.com) and the Eyetracker of the Foster-Miller, Inc., Waltmann, Mass. (www.foster-miller.com) are very useful for dynamic measurements. The above eyetracers have been originally designed as a helmet head set system only for eyes tracing, but not antropometry.

### SUMMARY

Existing methods and the apparatus' used for the determination of eyesight or eye tracking systems are sophisticated and expensive, need special software and specially trained specialists, and need substantial time to get results of a measurement, etc. At the same time, some special tools (“ergomeasure™”, Herman Miller, Inc., Zeeland, Mich.) that are designed for adjustment of office furniture are less expensive and less sophisticated compared with the apparatus' described above, but they are not precise enough.

Objects and Advantages

The determination of the eyesight's (line of eyesight) direction in the presented invention is based on the concept of a firearm's sight (for example rifle, gun, etc.). Thus, when we are looking through the firearm's rear back sight—“V” block and front bead, a virtual straight line from eye's pupil (lens) passing through the back sight is in the direction of our eyesight. The coordinates of this line determine the eyesight.

The presented apparatus and method is precise and inexpensive, compact and simple to use with immediate results of measure. The most of it application is in engineering anthropometry, operator work place design, architecture, out-door advertising (positioning billboard), etc. The special application of the patent is visible in an area when the PC user has to position the top of the VDT screen at the user's eye level while adjusting the PC workstation or lay out the VDT's in a dispatcher room.

### DRAWING AND PHOTOGRAPH FIGURES

The reference numerals in the drawing and photographs have slightly different numbers.

FIG. 1 shows the actual optical head (housing) that applied for patent.

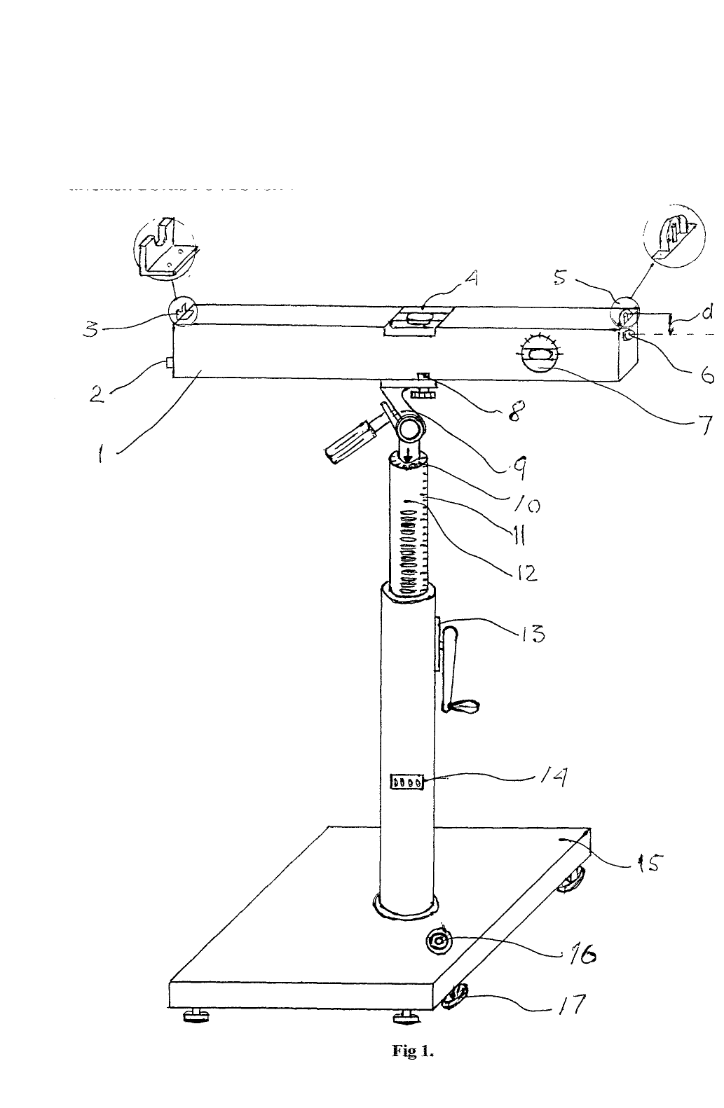

FIG. 2 shows a photograph of the optical head attached to manual moving mechanism.

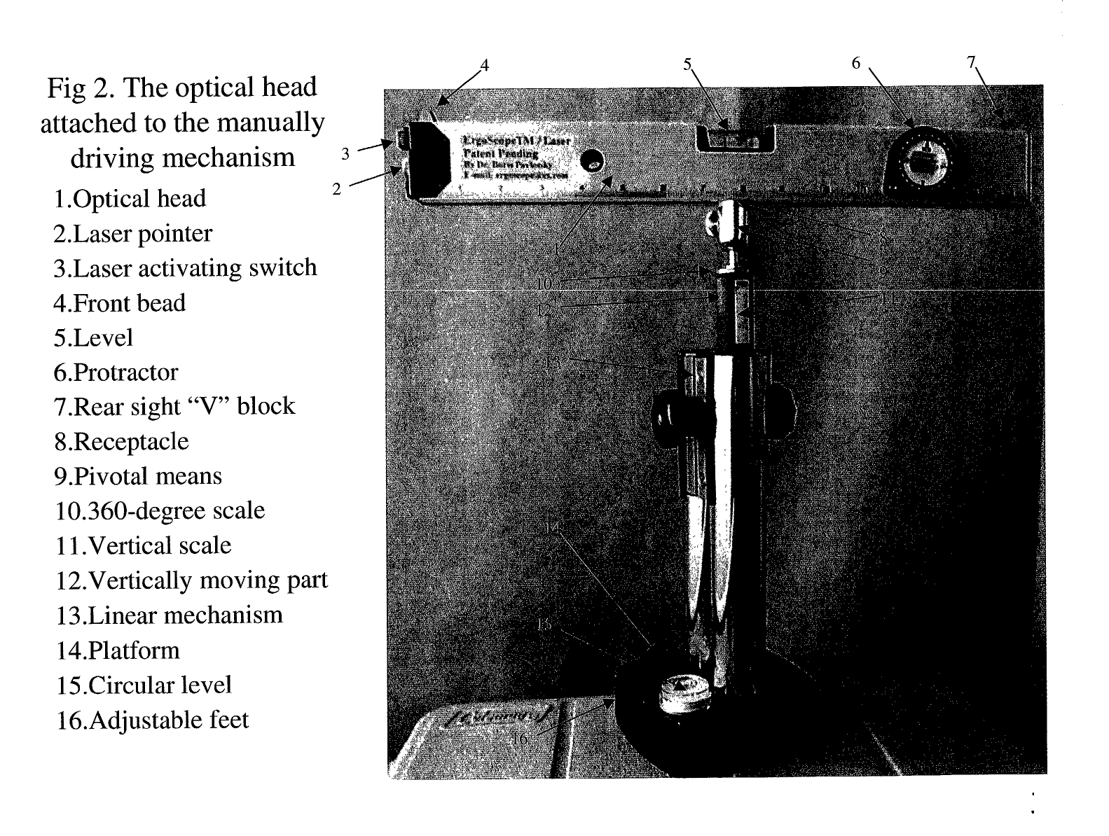

FIG. 3 shows a photograph of the optical head attached to motor moving mechanism and digital display of its vertical movement.

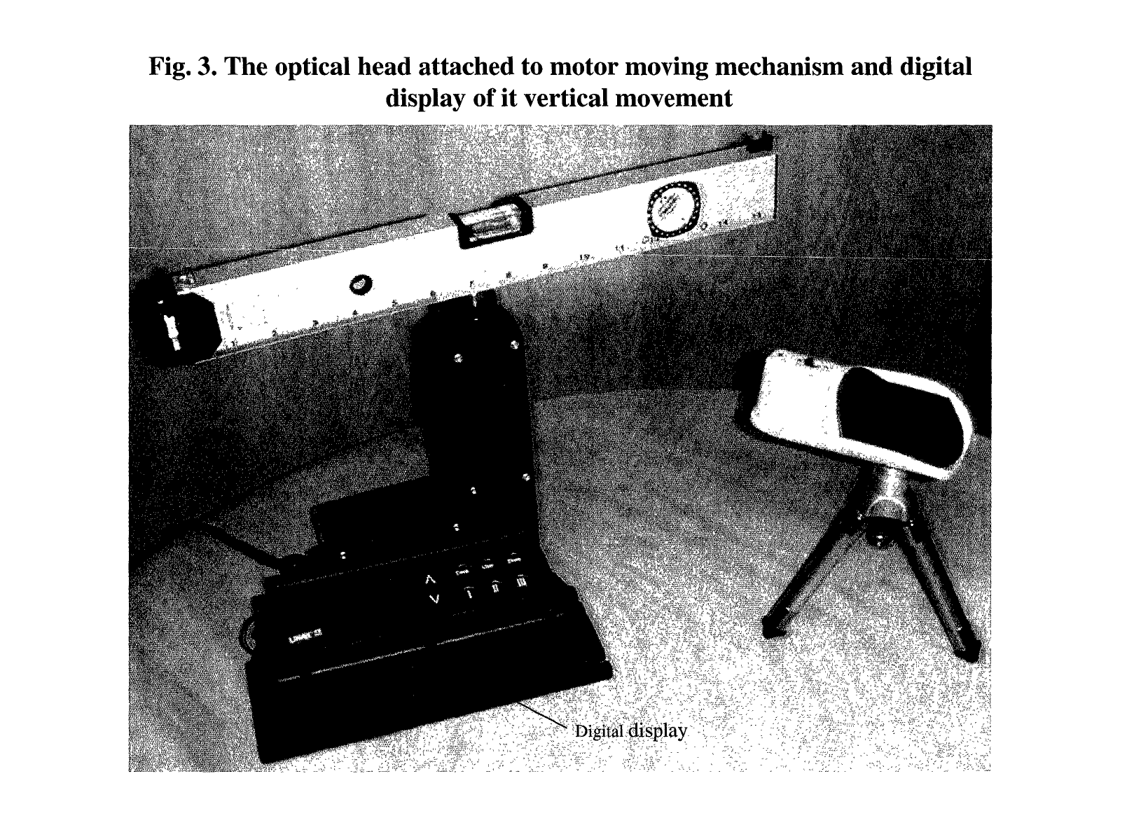

FIG. 4 shows a photograph detecting the PC user's eye level.

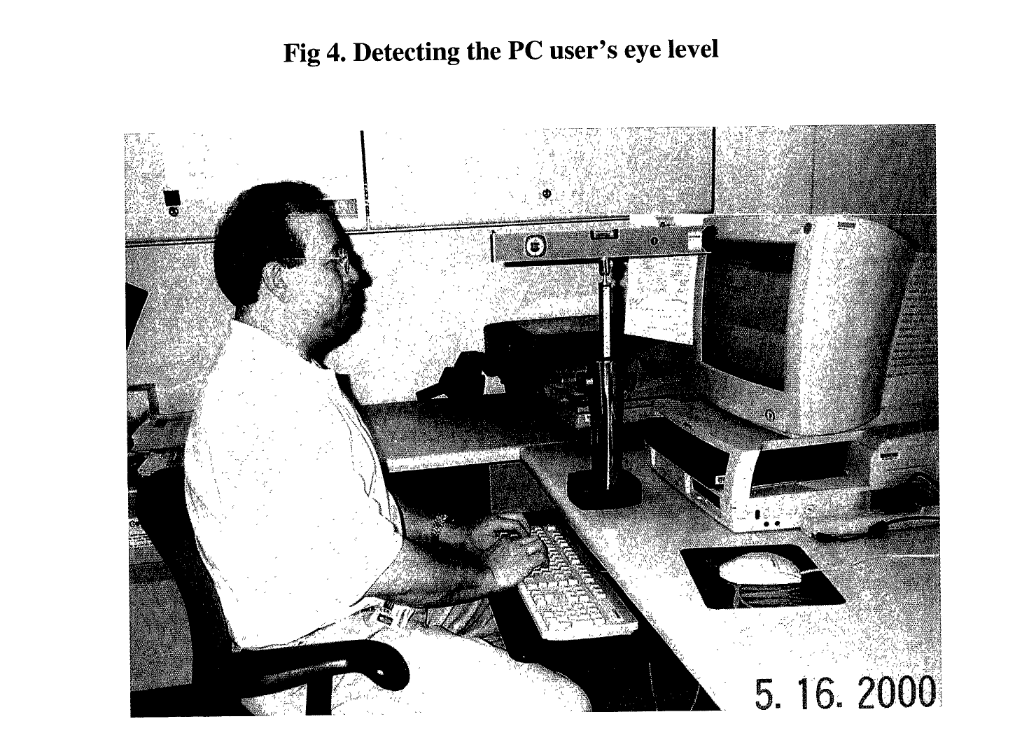

LIST OF REFERENCE NUMERALS IN THE DRAWING

```
  1 = Optical head (housing)
  2 = Laser activating switch
  3 = Rear sight “V” block
  4 = Level
  5 = Front bead
  6 = Laser pointer (laser beam)
  7 = Inclination measuring system (protractor)
  8 = Receptacle
  9 = Means for tilting and swiveling optical head
10 = 360° circular scale
11 = Vertical scale
12 = Vertically moving part
13 = Linear mechanism
14 = Digital display
15 = Platform
16 = Circular level
17 = Adjustable feet
d = Distance between top point of front bead and laser beam
(center of laser pointer)
```

### DESCRIPTION

The significant part of the apparatus is an optical head (a housing) 1 comprising a firearm's sight systems. For example, this is two elements—a rear sight “V” block 3 and a front bead 4 of the open-sight system of firearms and a level 4 to positioning the head in the horizontal level. A built-in inclination measuring system (protractor) 7 is used to read out angles of the optical head position/movement in a vertical plane. A laser pointer 6 with an activating switch 2 is functioning as an eye tracker and determines the gazing point. A built-in receptacle 8 joins pivotably the optical head (housing) 1 with tilting in the vertical plane and swiveling in the horizontal plane mechanism 9. A vertically moving part 12 of a linear driving mechanism 13 is moving the optical head 1 up or down. A vertical movement of the head is read-out on a scale 11 or on a digital display 14. Angles of the head movement in the horizontal plane are read-out on a 360-degree scale 10. The linear driving mechanism 13 is installed vertically on a platform 15. Adjustable feet 17 and a circular level 16 of the platform 15 are used for installing it in the horizontal plane.

Operation

There could be at least two circumstances (A, B) of measurements of eyesight's directions and angles within the interior/exterior space:

Circumstance A. There is a need to look at specific eyesight directions, for example a subject is asked to look straight horizontally.

A horizontal eyesight level is used as the reference point (Standard Sight Line) in anthropometric theory as eye height (while sitting or standing) for the designer of a vehicle's cabin or airliner's cockpit, etc. Another application is to properly position of a VDT (video display terminal) on a desk for the PC user. According to the ANSI recommendations, the top of the desktop PC monitor should be placed at eye level. So, the need is to determine horizontal eye level and point of view on the VDT screen.

Circumstance B. The person is gazing at a specific subject (area) during a task performance or just browsing within the interior/exterior space. There is a need to determine the angle of the eyesight's direction and the gazing point.

The measurement procedures in circumstances “A” and “B” are quite different.

The measurement procedure in circumstance “A” is as the follows:

1. The hypothetical PC user should be asked to relax and sit straight in a properly adjusted chair according to his or her anthropometric data.

2. The apparatus' support platform 15 should be installed in a horizontal position by adjustable feet 17 and checked by a circular level 16. Thus, the optical head's up and down moving part 12 is positioned vertically.

3. The optical head 1 is placed in the front of a PC user in the horizontal position. It is checked by the level 4 and moved up or down by the mechanism 13. The rear sight “V” block 3 should be positioned approximately at eye level and as close as possible to one of the eyes to look through (similar to targeting a firearm). One of the eyes should be covered up with an eye paddle.

4. The optical head 1 should be attuning (moving up or down) until the user looks straight through the rear sight's “V” block 3 and the front bead 5. Thereby this line will indicate a PC user's visual line direction or user's personal eye level in a sitting posture.

5. The distance of the eyesight's level from the desktops (or floor) will be read out on a vertical movement scale 11 or a digital display 14.

6. Activated by a switch 2, the laser pointer 6 will detect the user's gaze point on the VDT screen. Some corrections should be made considering the distance (d) between the top point of front bead and the laser beam (the center of the laser pointer).

The procedure for PC users in a standing posture will be the same.

The measurement procedure in circumstance “B”, when there is a need to determine the angle of the eyesight's direction and the gazing point, will be as follows:

1. The person is told to relax while gazing at a specific subject or area during a task performance or just browsing within the interior/exterior space in a sitting, standing or other posture position.

2. The optical head's platform 15 should be installed in the horizontal position by using adjustable feet 17 and checked by a circular level 16. Thus, the optical head's up and down moving part 12 is positioned vertically.

3. The optical head 1 is placed in the front of the person at approximately eye level and oriented at a direction of gazing a selected object or an area.

4. The rear sight “V” block 3 should be positioned as close as possible to the left or right eye to look through (similar to targeting a firearm). One of the eyes should be covered up with an eye paddle.

5. The optical head should be attuning (moving) by a linear driving mechanism 13 up or down and tilted or swiveled by mechanism 9 until the person looks straight through the rear sight's “V” block 3 and front bead 5 at viewing subject or area. Thereby the angle of this sight's line will indicate the person's eyesight direction while performing the task of gazing a selected object or a selected area in a sitting or standing posture position.

6. Activated by a switch 2, the laser pointer 6 will indicate the operator's gazing point of viewing the subject or the area. Some corrections should be made considering the distance (d) between the top point of the front bead and the laser beam (the center of the laser pointer).

I claim:

1. An apparatus and method for detecting eyesight direction, angle and gaze point comprising:
a housing supported, directed and moved by a supporting device or by hand(s)
a firearm's sight system located within said housing determines eyesight directions
an inclination measuring system located within said housing determines an angle of said eyesight
a laser pointer located within said housing determines said gazing point
